(19) United States
(12) Patent Application Publication
(10) Pub. No.: US 2014/0271639 A1
(43) Pub. Date: Sep. 18, 2014

(54) Semaphorin 7a Induced Lung Fibrogenesis Occurs in a CD-4-Dependent, Macrophage Dependent Manner

(71) Applicant: YALE UNIVERSITY, NEW HAVEN, CT (US)

(72) Inventor: Erica Herzog, New Haven, CT (US)

(21) Appl. No.: 14/349,885

(22) PCT Filed: Oct. 4, 2012
(86) PCT No.: PCT/US12/58703

§ 371 Date: Apr. 4, 2014

Related U.S. Application Data
(60) Provisional application No. 61/543,074, filed on Oct. 4, 2011.

(51) Int. Cl. C12N 15/113; C07K 14/705; C07K 16/18; C07K 16/28; C07K 14/47

(52) U.S. Cl. CPC C12N 15/1138; C07K 16/28; C12N 15/113; C07K 14/47; C07K 16/18; C07K 14/70596
USPC 424/133.1; 424/152.1; 424/172.1; 424/173.1; 514/44 R; 514/44 A

(57) ABSTRACT

The present invention provides methods of treating a mammal diagnosed with, or at risk for developing, pathogenic fibrosis, particularly pulmonary fibrosis. The method of the invention comprises administering to the mammal a compound or composition which modulates a target, such as Cofilin-1 and/or Plexin C1. The method of the invention also comprises administering to the mammal a compound or composition which inhibits a lymphocyte that expresses Sema 7a.

### CROSS-REFERENCE TO RELATED APPLICATIONS

This application claims the benefit of priority from U.S. Provisional Application Ser. No. 61/543,074, filed on Oct. 4, 2011, the entire disclosure of which is incorporated by reference herein as if set forth herein in its entirety.

### BACKGROUND OF THE INVENTION

Idiopathic Pulmonary Fibrosis is a chronic, progressive form of pulmonary fibrosis for which there is no known cure. The prevalence of this disease is increasing and affects millions of people worldwide (Homer et al., 2011, Arch. Pathol. Lab. Med. 135:780-788). Despite many years of research in this area, nearly all clinical trials have yielded negative results leaving IPF patients with few treatment options beyond supplemental oxygen and lung transplantation (Gen et al, Ther. Clin. Risk. Manag. 7:39-47). Thus, a better understanding of the factors regulating this disease is critical. Current paradigms for the development of IPF include recurrent or prolonged epithelial injury followed by inflammation and an abnormal wound healing response (Noble et al., 2004, Clin. Chest Med. 25:749-758). The immunologic factors promoting these effects remain unclear. While IPF's lack of responsiveness to corticosteroids implies that inflammation is not the primary event, the importance of inflammation in pulmonary fibrosis has clearly been shown in multiple animal models (Jiang et al., 2005, Nat. Med. 11:1173-1179). Recent work demonstrates that cells of monocyte lineage in the lungs and blood of patients with IPF display M2 markers, where they may predict disease progression (Murray et al., 2010, PLoS One 5:e9683), and animal modeling reveals that modulation of the M2 phenotype, or removal of intrapulmonary macrophages, results in attenuation of fibrosis in two separate models of lung fibrosis (Murray et al., 2010, PLoS. One 5:e9683; Murray et al., 2011, Int. J. Biochem. Cell Biol. 43:154-162). However the regulatory mechanisms causing these effects have not been completely defined.

The TGF-β1 superfamily proteins control such diverse biologic processes as cell growth and survival, cell and tissue differentiation, development, inflammation, immunity and tissue remodeling (Shi et al., 2003, Cell 113:685-700). TGF-β1 is essential for wound healing, stimulates matrix molecule deposition, and has been implicated in the pathogenesis of a variety of fibrotic disorders, including IPF (Noble et al., 2004, Clin. Chest Med. 25:749-758), scleroderma related interstitial lung disease (Dong et al., 2002, Proc. Nat. Acad. Sci. USA 99:3908-3913), and radiation-induced pulmonary fibrosis (Anscher et al., 1998, Oncol. Biol. Phys 41:1029-1035). TGF-β1 is secreted as a quiescent precursor that is activated through several mechanisms. This active form then binds to serine/threonine kinase receptors which activate SMAD2/3 phosphorylation to initiate gene transcription. TGF-β1 activity is inhibited by the endogenous inhibitor SMAD7 (Shi et al., 2003, Cell 113:685-700). A number of SMAD2/3 independent signaling pathways exist including one involving Semaphorin 7a (Lee et al., 2004, J. Exp. Med. 200:377-389). Animal modeling demonstrates that TGF-β1 is a critical mediator of bleomycin-induced pulmonary fibrosis (Breen et al., 1992, Am. J. Respir. Cell Mol. Biol. 6:146-152). It has been shown that lung-specific transgenic overexpression of the bioactive form of the human TGF-β1 gene induces a progressive fibrotic response characterized by a wave of epithelial apoptosis at 48 hours that resolves, monocytic inflammation that peaks at five days, dysregulated collagen production that occurs starting approximately day 10, and pronounced fibrosis by day 14 of transgene activation (Lee et al., 2004, J. Exp. Med. 200:377-389) that is accompanied by the robust intrapulmonary accumulation of CD206+ M2 macrophages (Lynne et al., 2009, in revision). While it has previously been shown that removal or modulation of these CD206+ cells via pharmacologic means attenuates experimentally induced lung fibrosis via SMAD-independent mechanisms (Murray et al., 2011, Int. J. Biochem. Cell Biol. 43:154-162), further understanding of the factors driving TGF-β1-induced M2 accumulation remain undefined.

The semaphorins (Semas) are a large family of highly conserved, secreted, and membrane-bound proteins that are divided into eight classes based on sequence similarities and distinct structural features (Eickholt, 2008, Development 135:2689-2694; Kumanogoh et al., 2003, J. Cell Sci. 116:3463-3470). Semas are expressed on nerve, myeloid, and lymphoid cells through which they regulate immune responses and such biologic processes as organogenesis, angiogenesis, apoptosis, and neoplasia (Holmes et al., 2002, Scand. J. Immunol. 56:270-275; Czopik et al., 2006, Immunity 24:591-600; Delorme et al., 2005, Biol. Cell 97:589-597). Semaphorin 7A (Sema 7a), also called CDw108, is a GPI anchored membrane protein that shares sequence similarities with the vaccinia virus Sema homologue A39R (Czopik et al., 2006, Immunity 24:591-600; Lange et al., 1998, Genmoics 51:340-350; Comeau et al., 1998, Immunity 8:473-482) and signals through at least two known receptors, β1 integrin and Plexin C1 (Kumanogoh et al., 2003, J. Cell Sci. 116:3463-3470). Sema 7a signaling through β1 integrin is required for proper axon track formation during embryonic development (Maruyama et al., 2008, Dev. Neurobiol. 68:317-331; Pasterkamp et al., 2003, Nature 424:680-684) and may contribute to inflammation, immunity, and osseous tissue responses based on its ability to stimulate macrophage chemotaxis and cytokine production (Suzuki et al., 2007, Nature 446:680-684), dendritic cell migration and chemokine expression (Czopik et al., 2006, Immunity 24:591-600), inhibit T cell function (Czopik et al., 2006, Immunity 24:591-600), stimulate osteoblast migration (Delorme et al., 2005, Biol. Cell 97:589-597), osteoclast fusion (Delorme et al., 2005, Biol. Cell 97:589-597) and promote melanocyte spreading and melanoma invasion (Lazova et al., 2009, Am. J. Dermatopathol. 31:177-181; Scott et al., 2008, J. Invest. Dermatol. 128:151-161) and regulate the production of collagen by fibrocytes (Gan et al., 2011, Arthritis Rheum. 63:2484-2494). Many of these latter effects are opposed by Plexin C1's effects on cytoskeletal rearrangement via the actin-modifying protein Cofilin-1 (CFL-1) (Czopik et al., 2006, Immunity 24:591-600; Lazova et al., 2009, Am. J. Dermatopathol. 31:177-181; Scott et al., 2008, J. Invest. Dermatol. 128:151-161; Gan et al., 2011, Arthritis Rheum. 63:2484-2494). Curiously, despite the known relationship between TGF-β1-induced M2 activation and IPF, a role for Semaphorin 7a and its downstream signaling pathways in this disease has not been explored.

The role of lymphocytes in experimental and human lung fibrosis remains controversial (Luzina et al., 2008, Leukoc. Biol. 83:237-244) and is only just beginning to be studied in earnest. CD4+ cells have been classically divided into TH1 and TH2 subgroups, with TH1 cells secreting IFNβ and TNF to drive cell-mediated immune effects, and TH2 cells secreting IL-4, IL-5, IL-6, IL-10 and IL-13 to promote humoral immune responses. This paradigm has been challenged by the discovery of TH17 cells, a CD4+ population that responds to IL-6 and TGF-β1 to secrete IL17 (which is associated with allergic responses and autoimmunity). TH2 and TH17 cells may participate in fibrosis through any number of mechanisms including secretion of soluble factors or contact-mediated membrane interactions that drive profibrotic effects on fibroblasts, macrophages, and epithelium. In this light, it is particularly significant that the lymphocyte attractant CCL18 demonstrates a remarkable correlation with disease severity in patients with IPF (Prasset et al., 2009, Am. J. Respir. Crit. Care Med. 179:717-723) and is sufficient to induce CD3-dependent fibrosis when overexpressed in the murine lung (Luzina et al., 2009, Arthritis Rheum. 60:1530-1539). It has also been suggested that Th17 cells promote fibrosis in an IL-17 dependent and IL-22 dependent manner (Wilson et al., J. Exp. Med. 207:535-552; Wilson et al., 2010, J. Immunol. 184:4378-4390). Direct evidence of an association between CD4+ lymphocytes and IPF is provided by a recent study demonstrating that expansion of a CD4+ population lacking both the costimulatory molecule CD28 and the regulatory molecule Fox P3 correlates negatively with disease outcome in patients with IPF (Gilani et al., 2010, PLoS One 5:e8959). Without being bound by any particular theory, these data suggest a previously unrecognized role for CD4+ lymphocytes in the development or maintenance of lung fibrosis. However the data are far from conclusive and the mechanism or mechanisms through which CD4+ cells may participate in fibrosis remain unclear. While Semaphorin 7a-expressing CD4+ cells are known to regulate macrophage activation in murine models of skin inflammation (Suzuki et al., 2007, Nature 446:680-684), a broader role for these cells in the development of M2 activation and fibrogenesis, as well as IPF, has been neither explored nor proposed.

There exists a need for better understanding of the role of Semaphorin 7a-expressing CD4+ cells and Cofilin-1 in the development of or maintenance of lung fibrosis, and in IPF in particular. The present invention addresses this unmet need.

### BRIEF SUMMARY OF THE INVENTION

The present invention relates to a method of inhibiting fibrosis in a mammal at risk of developing fibrosis. The present invention also relates to a method of inhibiting fibrosis in a mammal diagnosed with a disease or disorder involving fibrosis.

In one embodiment, the method comprises administering a therapeutically effective amount of a modulator of a target to the mammal, wherein the target is selected from the group consisting of Cofilin-1 (CFL-1), Plexin C1, and any combination thereof, thereby inhibiting fibrosis in the mammal. Preferably, the mammal is a human.

In one embodiment, the fibrosis comprises idiopathic pulmonary fibrosis.

In another embodiment, the modulator inhibits CFL-1.

In one embodiment, the modulator activates Plexin C1.

In yet another embodiment, the fibrosis comprises a pulmonary pathology selected from the group consisting of interstitial lung disease, scleroderma, radiation-induced pulmonary fibrosis, and bleomycin lung.

In yet another embodiment, the fibrosis comprises any pulmonary disorder wherein at least one of fibroproliferative matrix molecule deposition, enhanced pathological collagen accumulation, apoptosis and alveolar septal rupture with honeycombing occurs.

In yet another embodiment, the idiopathic pulmonary fibrosis is TGF-β1-induced.

In one embodiment, the modulator comprises an antibody, a soluble receptor of the target, an siRNA, a ribozyme, an antisense, an aptamer, a peptidomimetic, a small molecule, a soluble receptor, an agonist, or any combinations thereof.

In yet another embodiment, the antibody comprises an antibody selected from a polyclonal antibody, a monoclonal antibody, a humanized antibody, a synthetic antibody, a heavy chain antibody, a human antibody, and a biologically active fragment of an antibody.

In yet another embodiment, the biologically active fragment is a Fab fragment, and a F(ab′)₂ fragment, and combinations thereof.

In yet another embodiment, the heavy chain antibody is selected from the group consisting of a camelid antibody, a heavy chain disease antibody, and a variable heavy chain immunoglobulin.

In yet another embodiment, the antibody specifically binds to the target, a receptor of the target, a downstream effector of the target, or a functional fragment thereof.

The present invention also relates to a method of inhibiting fibrosis in a mammal at risk of developing fibrosis. In one embodiment, the method comprises administering a therapeutically effective amount of an inhibitor for a lymphocyte expressing Sema 7a to the mammal, thereby inhibiting fibrosis in the mammal. In one embodiment, the lymphocyte is a CD4 cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1E, depicts the effect of Sema 7a deletion on TGF-β1 induced lung inflammation, fibrosis, and M2 accumulation. FIG. 1A is a graph depicting how TGF-β1×Sema 7a−/− mice demonstrate unchanged BAL cell counts when compared to TGF-β1 mice in which the Sema 7a locus is intact (Sema 7a+/+). FIG. 1B is a graph depicting significantly reduced collagen accumulation in TGF-β1×Sema 7a−/− mice, as measured by Sircol analysis.

FIG. 1C is a series of photographs depicting significantly reduced collagen accumulation in TGF-β1×Sema 7a−/− mice as visualized using Masson's trichrome staining. FIGS. 1D and 1E depict the effect of Sema 7a deletion on TGF-β1-induced lung M2 accumulation. FIG. 1D is a graph depicting how the expression of the M2 related genes MRC/CD206 and MSR1 are significantly reduced, as measured in whole lung tissue RNA using quantitative real time PCR and normalized to β-actin housekeeping gene. FIG. 1E is a graph depicting the reduction in the accumulation of intrapulmonary macrophages expressing CD206 as measured by flow cytometry. n≧5 mice/group, 3 repetitions. *P≦0.05, **P≦0.01, ***P≦0.0001, ****P≦0.00001.

FIG. 2, comprising FIGS. 2A-2D, depicts the detection of Sema 7a in the human lung. FIG. 2A is a graph depicting how the elevated expression of Sema 7a, as measured by quantitative real time PCR, was detected in the lungs of IPF patients in comparison to healthy control lung tissue. FIG. 2B is a graph depicting the quantification of Sema 7a+ cells following immunostaining in lung tissue sections from healthy control lung (n=5) or IPF patients (n=5), which was also detected increased Sema 7a+ cells. FIG. 2C is a photograph depicting representative sections from control patient lung tissue immunostained for Sema 7a cells and counter stained with haematoxylin. FIG. 2D is a photograph depicting IPF patient lung tissue immunostained for Sema 7a cells and counter stained with haematoxylin. Sema 7a+ cells have the morphological appear of haematopoietic cells and appear brown (black arrow); with little to no detection observed in fibroblasts (red arrow), or lung epithelial cells (blue arrow). 60× original magnification. *P≦0.05, ****P≦0.00001.

FIG. 3, comprising FIGS. 3A-3G, depicts the detection of Sema 7a in human PBMCs. FIG. 3A is a graph depicting the increased expression of Sema 7a relative to GAPDH in the blood of IPF patients (n=23) as compared to normal control (n=34). FIG. 3B is a graph depicting the significantly increased percentage of Sema 7a+ CD19+ cells in IPF patients in comparison to healthy controls. FIG. 3C is a graph depicting the significantly increased percentage of Sema 7a+ CD19+ cells in IPF patients in comparison to healthy controls following flow cytometric analysis for CD19 (X axis) and Sema 7a (Y axis). FIG. 3D is a graph depicting significantly increased CD4+ Sema 7a+ cells in the circulation of IPF patients in comparison to healthy age matched controls. FIG. 3E is a graph depicting significantly increased CD4+ Sema 7a+ cells in the circulation of IPF patients following flow cytometric analysis. FIG. 3F is a graph depicting flow cytometric analysis of PBMCs from IPF patients with rapid (checked bar) or stable (black bar) disease indicating elevated CD4+ Sema 7a+ cells in patients with rapid IPF. FIG. 3G is a graph depicting flow cytometric analysis of PBMCs from IPF patients with rapid (checked bar) or stable (black bar) disease indicating increased percentages of circulating CD206+ CD14+ cells in patients with rapid IPF. *P≦0.05, **P≦0.01, ***P≦0.0001, ****P−0.00001.

FIG. 4, comprising FIGS. 4A-4E, depicts how bone marrow-derived Sema 7a+ cells promote fibrosis and M2 macrophage accumulation in the lung. FIG. 4A is a graph depicting that the reconstitution of Sema 7a−/− mice with bone marrow from Sema 7a+/+ mice had no effect on Bal cell accumulation. FIG. 4B is a graph depicting that the reconstitution of Sema 7a−/− mice with bone marrow from Sema 7a+/+ mice restored the TGF-β1-induced lung fibrotic response as measured by total soluble collagen quantitated using the Sircol assay. FIG. 4C is a graph depicting CD206+ cells in the BAL quantified by flow cytometry. FIG. 4D is a series of photographs depicting collagen deposition visualized using Masson's Trichrome staining FIG. 4E is a graph depicting that the depletion of intrapulmonary monocytes/macrophages with intranasal clodronate delivery prior to TGF-β1 Tg activation further inhibited collagen deposition, as measured using the Sircol assay, in Sema 7a−/− mice that had received Sema 7a+/+ bone marrow. n≧5 mice/group, 3 repetitions. *P≦0.05, ****P≦0.00001.

FIG. 5, comprising FIGS. 5A-5G, depicts that CD4-deficient and not CD19-deficient bone marrow derived cells recapitulate Sema 7a deficient responses in TGFβ Tg mice. FIGS. 5A-5B depict that in the TGF-β1-exposed murine, Sema 7a+CD19+ cells are very rare while there is robust detection of Sema 7a+ CD4+ cells. Nuclei are counterstained with DAPI (blue). FIG. 5A is a photograph depicting the immunofluorescent colocalization of single cell suspensions from TGFβ Tg mice of Sema 7a (green) and CD19 (red). FIG. 5B is a photograph depicting the immunofluorescent colocalization of single cell suspensions from TGFβ Tg mice of Sema 7a (green) and CD4 (red). FIG. 5C is a graph depicting the effects of donor bone marrow on lung fibrosis after 14 days of TGFβ Tg activation on BAL cell accumulation. FIG. 5D is a graph depicting the effects of donor bone marrow on lung fibrosis after 14 days of TGFβ Tg activation on lung CD206+ cell number as determined by flow cytometry. FIG. 5E is a graph depicting the effects of donor bone marrow on lung fibrosis after 14 days of TGFβ Tg activation on lung collagen as measured by Sircol assay. FIG. 5F is a series of photographs depicting lung collagen deposition as visualized using Masson's Trichrome staining. FIG. 5G is a graph depicting the effects of donor bone marrow on lung fibrosis after 14 days of TGFβ Tg activation on lung CXCL10/IP10 expression quantitated using real time PCR. TGFβ Tg+ Sema 7a−/− mice were reconstituted with donor bone marrow from wildtype, CD4−/−, CD19−/− or Sema 7a−/− mice. n≧5 mice/group, 2 repetitions. *P≦0.05, **P≦0.01, ****P≦0.00001.

FIG. 6, comprising FIGS. 6A-6E, depicts the effects of Sema 7a and Sema 7a receptor inhibition on primary monocytes. FIG. 6A is a graph depicting the stimulation of CD 14+ monocytes from control (n=8) and IPF patients (n=8) with recombinant Sema 7a and the determination of the effect on CD206 expression using flow cytometry. FIG. 6B is a graph depicting a quantification of the stimulation of CD14+ monocytes from control (n=8) and IPF patients (n=8) with recombinant Sema 7a and the determination of the effect on CD206 expression. FIG. 6C is a graph depicting the increased expression in the blood of IPF patients of the Sema 7a receptors CFL1. FIG. 6D is a graph depicting the increased expression in the blood of IPF patients of the Sema 7a receptors Plexin C1. For FIGS. 6C and 6D, the results were determined in lung tissue and circulating cell pellet RNA from control (open bars) and IPF patients (closed bars) using quantitative real time PCR. The expression levels of the gene of interest were normalized to β-actin (for CFL-1) or GAPDH (for Plexin C1). FIG. 6E is a graph depicting that lentivirally-mediated transfer of ShRNA against Plexin C1 leads to increased Sema 7a-mediated expression of CD206 in IPF but not normal monocytes. *P^{˜}0.05, **P^{˜}0.01.

FIG. 7, comprising FIGS. 7A-7B, depicts that elevated Cofilin-1 expression is macrophage- and fibrosis-associated during TGFβ-induced lung fibrosis. FIG. 7A is a series of photographs depicting the immunofluorescent colocalization of CFL-with F4/80+ macrophages observed in cytospins of BAL cells obtained from the TGF-β1-exposed murine lung. FIG. 7B is a graph depicting the restoration of TGFβ Tg fibrosis in Sema 7a−/− mice reconstituted with Sema 7a+/+ bone marrow, which was associated with an increase in lung CFL-1 expression, as measured by real time PCR (B). n≧5 mice/group. ***P≦0.001.

FIG. 8 is a photograph depicting how the immunostaining of Sema 7a on human lung tissue in the absence of primary antibody reveals no staining.

FIG. 9, comprising FIGS. 9A-9F, depicts no difference in Sema 7a expression on CD14+ cells or CD8+ cells between healthy controls and IPF patients.

FIG. 9A is a graph depicting the flow cytometric detection of CD14 (X axis) and Sema 7a (Y axis) in control samples. FIG. 9B is a graph depicting the flow cytometric detection of CD14 (X axis) and Sema 7a (Y axis) in IPF samples. FIG. 9C is a graph depicting how the flow cytometric detection of CD14 and Sema 7a in control versus IPF subjects reveals no significant difference in Sema 7a+CD14+ cells between these subjects. FIG. 9D is a graph depicting flow cytometric detection of CD8 (X axis) and Sema 7a (Y axis) in control samples. FIG. 9E is a graph depicting depicting flow cytometric detection of CD8 (X axis) and Sema 7a (Y axis) in IPF samples. FIG. 9F is a graph depicting how flow cytometric detection of CD8 and Sema 7a in control versus IPF subjects reveals no significant difference in Sema 7a+ CD14+ cells between these subjects.

FIG. 10, comprising FIGS. 10A-10C, depicts elevated CD206+ monocytes in the circulation of IPF patients. FIG. 10A is a graph depicting flow cytomteric detection of CD14 (X axis) and CD206 (Y axis) in control samples. FIG. 10B is a graph depicting flow cytomteric detection of CD 14 (X axis) and CD206 (Y axis) in IPF samples. FIG. 10C is a graph depicting significant increases in CD206+ CD14+ cells in the subjects with IPF.

### DETAILED DESCRIPTION

The present invention relates to the discovery that Sema 7a-expressing lymphocytes have a role in the induction of M2 activation and fibrogenesis in the TGF-β1 pathway. In one embodiment, the Sema 7a-expressing lymphocyte is a CD4 cell. The present invention also relates to the discovery that the actin modifying protein Cofilin-1 (CFL-1) has a role in controlling M2 activation in fibrogenesis. The present invention provides inhibitors of targets of the present invention, receptors of targets of the present invention, and downstream effectors of targets of the present invention to prevent, treat or reverse TGF-β1 induced DNA injury, cellular apoptosis, pathological deposition of collagen, alveolar remodeling, and fibrosis pathogenesis. In one embodiment, a target of the present invention is CFL-1. In another embodiment, a target of the present invention is a lymphocyte expressing Sema 7a. In a further embodiment, the lymphocyte is a CD4 cell.

In another embodiment, the invention provides compositions and methods for activating Plexin C1 in order to inhibiting fibrosis in a mammal

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document. As used herein, each of the following terms has the meaning associated with it in this section.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

Signal transduction is any process by which a cell converts one signal or stimulus into another, most often involving ordered sequences of biochemical reactions carried out within the cell. The number of proteins and molecules participating in these events increases as the process eminates from the initial stimulus resulting in a “signal cascade.” The phrase “downstream effector”, as used herein, refers to a protein or molecule acted upon during a signaling cascade, which in term acts upon another protein or molecule. The term “downstream” indicates the direction of the signaling cascade.

A disease or disorder is “alleviated” if the severity of a symptom of the disease, or disorder, the frequency with which such a symptom is experienced by a patient, or both, are reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

By the term “specifically binds,” as used herein, is meant a molecule, such as an antibody, which recognizes and binds to another molecule or feature, but does not substantially recognize or bind other molecules or features in a sample.

As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The phrase “inhibitor of a target of the present invention,” as used herein, refers to a composition or compound that inhibits the activity of a target of the present invention, either directly or indirectly, using any method known to the skilled artisan. An inhibitor of a target of the present invention may be any type of compound, including but not limited to, a polypeptide, a nucleic acid, an aptamer, a peptidometic, and a small molecule.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention is based on the discovery that Sema 7a is a robust inducer of M2 activation and the development of fibrosis. Specifically, it has been discovered that CD4 cells expressing Sema 7a play a role in M2 activation in lung fibrosis. The present invention also demonstrates that the pro-fibrotic effects of Sema 7a occur, at least in part, via a macrophage dependent mechanism. It has also been demonstrated that disease progression is predicted both by Sema 7a expressing CD4+ cells and expression of CD206 on circulating monocytes. The present invention also demonstrates that Plexin C1, the cognate receptor of Sema 7a and a Cofilin-1 inactivator, is increased in the circulation of IPF patients, suggesting a role for Plexin C1 in the TGF-β1 pathway of fibrogenesis. It has also been demonstrated that the effects of fibrosis are accompanied by increased levels of the actin-modifying protein Cofilin-1, suggesting a role of Cofilin-1 in the TGF-β1 pathway of fibrogenesis. It has also been demonstrated that CD4 lymphocytes are required for Sema 7a-expressing BMDC to induce TGF-β1-induced M2 accumulation and fibrosis.

Accordingly, the invention provides a method of inhibiting fibrosis in a subject diagnosed with a disease or disorder involving fibrosis or in a subject at risk of developing fibrosis.

In one embodiment, the invention provides a method of inhibiting fibrosis in a mammal at risk of developing fibrosis by administering a therapeutically effective amount of a modulator of a target to said subject, wherein the target is selected from the group consisting of Cofilin-1 (CFL-1) and Plexin C1, thereby inhibiting fibrosis in the mammal. In one embodiment, the modulator of CFL-1 is an inhibitor of CFL-1. In one embodiment, the modulator is an activator of Plexin C1.

A target of the present invention may be SEMA 7A receptors, SEMA 7A downstream effectors, such as Cofilin-1, or upstream regulators which up-regulate SEMA 7A expression. A target of the present invention may be lymphocytes expressing Sema 7a. In one embodiment, a target of the present invention is Cofilin-1 (CFL-1). In another embodiment, a target of the present invention is Plexin C1. In another embodiment, a target of the present invention is a lymphocyte expressing Sema 7a. In a further embodiment, the lymphocyte expressing Sema 7a is a CD4 cell.

The methods of the invention further comprise administering a therapeutically effective amount of a modulator of the target of the present invention wherein fibrosis is prevented, halted, or reversed in the subject. In one embodiment, the modulator inhibits CFL-1. In another embodiment, the modulator activates Plexin C1. In another embodiment, the modulator inhibits a lymphocyte expressing Sema 7a. In a further embodiment, the lymphocyte is a CD4 cell. In another aspect, the modulator of a lymphocyte expressing Sema 7a is comprised of a multiple binding domains, wherein at least one binding site is selective for Sema 7a and at least one binding domain is selective for CD4 cells.

The invention may be practiced in any subject diagnosed with, or at risk of developing, fibrosis. Fibrosis is associated with many diseases and disorders. Preferably, the fibrosis is idiopathic pulmonary fibrosis. The subject may be diagnosed with, or at risk for developing interstitial lung disease including idiopathic pulmonary fibrosis, scleroderma, radiation-induced pulmonary fibrosis, bleomycin lung, sarcoidosis, silicosis, familial pulmonary fibrosis, an autoimmune disease or any disorder wherein one or more fibroproliferative matrix molecule deposition, enhanced pathological collagen accumulation, apoptosis and alveolar septal rupture with honeycombing occurs. The subject may be identified as having fibrosis or being at risk for developing fibrosis because of exposure to asbestos, ground stone and metal dust, or because of the administration of a medication, such as bleomycin, busulfon, pheytoin, and nitro furantoin, which are risk factors for developing fibrosis. Preferably, the subject is a mammal and more preferably, a human. It is also contemplated that the compositions and methods of the invention may be used in the treatment of organ fibrosis secondary to allogenic organ transplant, e.g., graft transplant fibrosis. Non-limiting examples include renal transplant fibrosis, heart transplant fibrosis, liver transplant fibrosis, etc.

Modulating the activity of a target of the present invention can be accomplished using any method known to the skilled artisan. Examples of methods to modulate the activity of a target of the present invention include, but are not limited to modulating expression of an endogenous gene of a target of the present invention, modulating expression of the mRNA of a target of the present invention, and modulating activity of a protein of a target of the present invention. A modulator of a target of the present invention may therefore be a compound or composition that modulates expression of a gene of a target of the present invention, a compound or composition that decreases mRNA half-life, stability and/or expression, or a compound or composition that modulates the protein function of a target of the present invention. A modulator of a target of the present invention may be any type of compound, including but not limited to, a polypeptide, a nucleic acid, an aptamer, a peptidometic, and a small molecule, or combinations thereof.

Modulation of a target of the present invention may be accomplished either directly or indirectly. For example, a target of the present invention may be directly modulated by compounds or compositions that directly interact with the protein of a target of the present invention, such as antibodies or soluble receptors of a target of the present invention.

Modulating expression of an endogenous gene of a target of the present invention includes providing a specific modulator of the gene expression of a target of the present invention. Modulating expression of mRNA of a target of the present invention or protein of a target of the present invention includes modulating the half-life or stability of mRNA of a target of the present invention or modulating expression of mRNA of a target of the present invention. Methods of modulating expression of a target of the present invention include, but are not limited to, methods that use an siRNA, a microRNA, an antibody, a soluble receptor, an antisense nucleic acid, a ribozyme, an expression vector encoding a transdominant negative mutant, a peptide, a small molecule, other specific inhibitors of a gene of a target of the present invention, mRNA, and protein expression, and combinations thereof.

Sema 7a plays a critical role in the pathogenesis of TGF-β1-induced fibrosis and alveolar remodeling and that TGF-β1 regulates the expression of expression of Sema 7A receptors, extracellular matrix (ECM) proteins, proteases, anti-proteases, transcription factors, fibrogenetic cytokines, apoptosis regulators and IL-13 receptors via Smad 2/3-independent, Sema 7A-dependent activation pathways, as demonstrated by Elias et al., U.S. patent application Ser. No. 12/604,192, hereby incorporated by reference in its entirety.

### Methods of Inhibiting a Target of the Present Invention, a Receptor of a Target of the Present Invention, and Downstream Effector Protein: Antibodies

In one embodiment of the invention, the modulator of a target of the present invention is an antibody. It will be appreciated by one skilled in the art that an antibody comprises any immunoglobulin molecule, whether derived from natural sources or from recombinant sources, which is able to specifically bind to an epitope present on a target molecule. In the present invention, the target molecule may be a target of the present invention, a receptor of a target of the present invention, a downstream effector of a target of the present invention, or fragments thereof. In one embodiment, the antibody is specific for CFL-1. In another embodiment, the antibody is specific for a lymphocyte which expresses Sema 7a. In a further embodiment, the lymphocyte is a CD4 cell. In one aspect of the invention, a target of the present invention is directly inhibited by an antibody that specifically binds to an epitope on a target of the present invention. In another aspect of the invention, a target of the present invention is indirectly inhibited by an antibody that specifically binds to an epitope on a receptor of a target of the present invention receptor. In yet another aspect of the invention, the effects of a target of the present invention are blocked by an antibody that specifically binds to an epitope on a downstream effector such as extracellular matrix (ECM) proteins, proteases, anti-proteases, transcription factors, fibrogenetic cytokines, apoptosis regulators and IL-13 receptor components.

When the inhibitor of a target of the present invention used in the compositions and methods of the invention is a polyclonal antibody (IgG), the antibody is generated by inoculating a suitable animal with a peptide comprising the full length of a target of the present invention, a receptor of a target of the present invention, a downstream effector of a target of the present invention, or fragments thereof. These polypeptides, or fragments thereof, may be obtained by any method known in the art, including chemical synthesis and biological synthesis, as described elsewhere herein. Antibodies produced in the inoculated animal which specifically bind to a target of the present invention, a receptor of a target of the present invention, a downstream effector of a target of the present invention, or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against a full length target of the present invention, a receptor of a target of the present invention, a downstream effector of a target of the present invention, or fragment thereof, may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3, 4):125-168) and the references cited therein.

When the antibody used in the methods of the invention is a biologically active antibody fragment or a synthetic antibody corresponding to antibody to a full length target of the present invention, a receptor of a target of the present invention, a downstream effector of a target of the present invention, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3, 4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the target molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as inhibitors of a target of the present invention in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York (2002)). Exemplary immunoassays are described briefly below (but are not intended to be in any way limiting).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 14 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. Those of ordinary skill in the art will be knowledgeable as to the parameters that can be modified to increase the binding of the antibody to an antigen and decrease the background (e.g., pre-clearing the cell lysate with sepharose beads), upon consideration of the present disclosure. Additional immunoprecipitation protocols are presented Current Protocols in Molecular Biology, (Ausubel et al., eds.), Greene Publishing Associates and Wiley-Interscience, New York (2002).

Western blot analysis generally comprises preparing protein samples, electrophoresis of the protein samples in a polyacrylamide gel (e.g., about 8 20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with about 3% BSA or non-fat milk), washing the membrane in washing buffer (e.g., PBS-TWEEN 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. Those of ordinary skill in the art will be knowledgeable as to the parameters that can be modified to increase the signal detected and to reduce the background noise, upon consideration of the present disclosure. Additional western blot protocols are presented in Current Protocols in Molecular Biology, (Ausubel et al., eds.), Greene Publishing Associates and Wiley-Interscience, New York (2002).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. When performing an ELISA, the antibody of interest does not need to be conjugated to a detectable compound; instead, a second antibody (which recognizes the antibody of interest) conjugated to a detectable compound can be added to the well. Further, instead of coating the well with the antigen, the antibody may be coated to the well. In this case, a second antibody conjugated to a detectable compound can be added following the addition of the antigen of interest to the coated well. One of ordinary skill in the art will be knowledgeable as to the parameters that can be modified to increase the signal detected as well as other variations of ELISA protocols known in the art. For further discussion regarding ELISA protocols see, e.g., Current Protocols in Molecular Biology, (Ausubel et al., eds.), Greene Publishing Associates and Wiley-Interscience, New York (2002).

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

### Soluble Receptors

In another embodiment of the invention, soluble receptor protein of a target of the present invention that binds to of a target of the present invention is contemplated as an inhibitor of a target of the present invention. This agent can be used to reduce or prevent the binding of a target of the present invention to cell bound soluble receptor protein of a target of the present invention and thereby act as an antagonist of a target of the present invention. Soluble receptors have been used to bind cytokines or other ligands to regulate their function (Thomson, (1998) Cytokine Handbook, Academic Press). A soluble receptor occurs in solution, or outside of the membrane. Soluble receptors may occur because the segment of the molecule which spans or associates with the membrane is absent. This segment is commonly referred to in the art as the transmembrane domain of the gene, or membrane binding segment of the protein. Thus, in some embodiments of the invention, a soluble receptor includes a fragment or an analog of a membrane bound receptor. Preferably, the fragment contains at least six, e.g., ten, fifteen, twenty, twenty-five, thirty, forty, fifty, sixty, or seventy amino acids, provided it retains its desired activity.

In other embodiments of the invention, the structure of the segment that associates with the membrane is modified (e.g., DNA sequence polymorphism or mutation in the gene) so the receptor is not tethered to the membrane, or the receptor is inserted, but is not retained within the membrane. Thus, a soluble receptor, in contrast to the corresponding membrane bound form, differs in one or more segments of the gene or receptor protein that are important to its association with the membrane.

The present invention encompasses cDNA encoding a soluble receptor protein of a target of the present invention which is isolated from soluble receptor producing cells of a target of the present invention or is recombinantly engineered from DNA encoding soluble receptor producing cells of a target of the present invention. Soluble receptor producing cells of a target of the present invention, as used herein, refer to a protein which can specifically bind to a target of the present invention without eliciting undesired downstream effects including, but not limited to, pulmonary epithelial cell DNA destruction, apoptosis, pathological collagen deposition, and alveolar remodeling.

Sema 7a receptors known in the art include human Plexin C1, (GenBank Accession No.: NM_{—}005761) and human β1 integrin (GenBank Accession No.: P05556). However, the invention should not be considered to be limited to the use of these receptors. Any receptor of a target of the present invention identified may serve as the basis for the generation of a soluble receptor of a target of the present invention.

Any of a variety of procedures may be used to molecularly clone cDNA of soluble receptors of a target of the present invention. These methods include, but are not limited to, direct functional expression of the gene of the soluble receptor of a target of the present invention following the construction of soluble receptor of a target of the present invention-containing cDNA library in an appropriate expression vector system.

It is readily apparent to those skilled in the art that suitable cDNA libraries may be prepared from cells or cell lines which have activity for a soluble receptor of a target of the present invention. The selection of cells or cell lines for use in preparing a cDNA library to isolate cDNA of a receptor of a target of the present invention may be done by first measuring the activity of a receptor of a target of the present invention using a binding assay of a target of the present.

Preparation of cDNA Libraries can be performed by Standard Techniques Well known in the art. Well known cDNA library construction techniques can be found for example, in Maniatis, T., Fritsch, E. F., Sambrook, J., Molecular Cloning: A Laboratory Manual (Cold Spring Harbor Laboratory, Cold Spring Harbor, N.Y., 1982). It is also readily apparent to those skilled in the art that DNA encoding SEMA 7A-R may also be isolated from a suitable genomic DNA library. Construction of genomic DNA libraries can be performed by standard techniques well known in the art. Well known genomic DNA library construction techniques can be found in Maniatis, T., Fritsch, E. F., Sambrook, J. in Molecular Cloning: A Laboratory Manuel (Cold Spring Harbor Laboratory, Cold Spring Harbor, N.Y., 1982).

Molecules of cDNA of a soluble receptor of a target of the present invention may also be obtained by recombinantly engineering them from DNA encoding the partial or complete amino acid sequence of a receptor of a target of the present invention. Using recombinant DNA techniques, DNA molecules are constructed which encode at least a portion of the receptor of a target of the present invention capable of binding a target of the present invention without stimulating pulmonary endothelial cell DNA destruction, apoptosis, pathological collagen deposition, and alveolar remodeling. Standard recombinant DNA techniques are used such as those found in Maniatis, et al., supra.

DNA encoding a soluble receptor of a target of the present invention is constructed from a DNA sequence encoding a receptor of a target of the present invention. For purposes of illustration, DNA encoding the Sema 7a receptor plexin C1 is utilized. Using the receptor DNA sequence, a DNA molecule is constructed which encodes the extracellular domain of the receptor, or the Sema 7a binding domain only and is denoted sSema 7a-R. Restriction endonuclease cleavage sites are identified within the receptor DNA and can be utilized directly to excise the extracellular-encoding portion. In addition, PCR techniques well known in the art may be utilized to produce the desired portion of DNA. It is readily apparent to those skilled in the art that other techniques, which are standard in the art, may be utilized to produce molecules of cDNA of a soluble receptor of a target of the present invention in a manner analogous to those described above. Such techniques are found, for example, in Maniatis et al., supra.

The cloned sSEMA 7A-R cDNA obtained through the methods described above may be recombinantly expressed by molecular cloning into an expression vector containing a suitable promoter and other appropriate transcription regulatory elements, and transferred into prokaryotic or eukaryotic host cells to produce recombinant sSEMA 7A-R. Techniques for such manipulations are fully described in Maniatis, T, et al., supra, and are well known in the art.

Expression vectors are defined herein as DNA sequences that are required for the transcription of cloned copies of genes and the translation of their mRNAs in an appropriate host. Such vectors can be used to express eukaryotic genes in a variety of hosts such as bacteria, bluegreen algae, fungal cells, yeast cells, plant cells, insect cells and animal cells.

Specifically designed vectors allow the shuttling of DNA between hosts such as bacteria-yeast or bacteria-animal or bacteria-insect cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

A variety of mammalian expression vectors may be used to express recombinant a soluble receptor of a target of the present invention in mammalian cells. Commercially available mammalian expression vectors which may be suitable for recombinant expression, include but are not limited to, pMC lneo (Stratagene), pXT1 (Stratagene), pSGS (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and gZD35 (ATCC 37565).

DNA encoding a soluble receptor of a target of the present invention may also be cloned into an expression vector for expression in a recombinant host cell. Recombinant host cells may be prokaryotic or eukaryotic, including but not limited to bacteria, yeast, mammalian cells including but not limited to cell lines of human, bovine, porcine, monkey and rodent origin, and insect cells including but not limited to drosophila, moth, mosquito and armyworm derived cell lines. Cell lines derived from mammalian species which may be suitable and which are commercially available, include but are not limited to, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171). Insect cell lines which may be suitable and are commercially available include but are not limited to 3M-S (ATCC CRL 8851) moth (ATCC CCL 80) mosquito (ATCC CCL 194 and 195; ATCC CRL 1660 and 1591) and armyworm (Sf9, ATCC CRL 1711).

The expression vector may be introduced into host cells via any one of a number of techniques including but not limited to transformation, transfection, liposome or protoplast fusion, and electroporation. The expression vector-containing cells are clonally propagated and individually analyzed to determine whether they produce protein of a soluble receptor of a target of the present invention. Identification of a soluble receptor of a target of the present invention expressing host cell clones may be done by several means, including but not limited to immunological reactivity with anti-soluble receptor of a target of the present invention antibodies, binding to radiolabelled soluble receptor of a target of the present invention, and the presence of host cell-secreted activity of a soluble receptor of a target of the present invention.

Expression of DNA of a soluble receptor of a target of the present invention may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes.

Levels of protein of a soluble receptor of a target of the present invention produced by host cells may be quantitated by immunoaffinity and/or ligand affinity techniques. Affinity beads specific for a soluble receptor of a target of the present invention or antibodies specific for a soluble receptor of a target of the present invention are used to isolate ³⁵S-methionine labeled or unlabeled protein of a soluble receptor of a target of the present invention. Labeled protein of a soluble receptor of a target of the present invention is analyzed by SDS-PAGE. Unlabeled protein of a soluble receptor of a target of the present invention is detected by Western blotting, ELISA or RIA assays employing antibodies specific for a soluble receptor of a target of the present invention, or by ligand blotting with labeled target of the present invention.

Following expression of a soluble receptor of a target of the present invention in a recombinant host cell, protein of a soluble receptor of a target of the present invention may be recovered to provide a soluble receptor of a target of the present invention in active form, capable of binding a target of the present invention without stimulating lung epithelial cell DNA damage, apoptosis, pathological collagen deposition, and alveolar remodeling. Several purification procedures for a soluble receptor of a target of the present invention are available and suitable for use. A soluble receptor of a target of the present invention may be purified from cell lysates and extracts, or from conditioned culture medium, by various combinations of, or individual application of salt fractionation, ion exchange chromatography, size exclusion chromatography, hydroxylapatite adsorption chromatography, reversed phase chromatography, heparin sepharose chromatography, ligand affinity chromatography for a soluble receptor of a target of the present invention, and hydrophobic interaction chromatography.

In addition, recombinant soluble receptor of a target of the present invention can be separated from other cellular proteins by use of an immuno-affinity column made with monoclonal or polyclonal antibodies specific for full length soluble receptor of a target of the present invention, or polypeptide fragments of a soluble receptor of a target of the present invention. The protein of a soluble receptor of a target of the present invention can be expressed using a baculovirus expression system. The recombinantly produced soluble receptor of a target of the present invention is purified from the recombinant host cell extracts or cell culture fluid using heparin-sepharose column chromatography which specifically binds the protein of a soluble receptor of a target of the present invention. The heparin-sepharose bound soluble receptor of a target of the present invention column is washed using a suitable buffer containing between b 0.1M and 0.6M NaCl which removes contaminating proteins without significant loss of soluble receptor of a target of the present invention. The soluble receptor of a target of the present invention is eluted from the heparin-sepharose column using a suitable buffer containing about 1M NaCl, yielding substantially purified soluble receptor of a target of the present invention.

siRNA

In one embodiment, siRNA is used to decrease the level of protein of a target of the present invention. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(Comeau et al., 1998, Immunity 8:473-482):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of modulating levels of SEMA 7A protein using RNAi technology.

Modification of siRNA

Following the generation of the siRNA polynucleotide of the present invention, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

### Vectors

In other related aspects, the invention includes an isolated nucleic acid encoding an inhibitor, wherein the inhibitor such as an siRNA, inhibits a protein of a target of the invention, a receptor of a target of the present invention, a regulator thereof; or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). In another aspect of the invention, a target of the present invention, a receptor of a target of the present invention, or a regulator thereof, can be inhibited by way of inactivating and/or sequestering a target of the present invention, a receptor of a target of the present invention, or a regulator thereof. As such, inhibiting the effects of a target of the present invention can be accomplished by using a transdominant negative mutant.

In another aspect, the invention includes a vector comprising a siRNA polynucleotide. Preferably, the siRNA polynucleotide is capable of inhibiting the expression of a target polypeptide, wherein the target polypeptide is selected from the group consisting of a target of the present invention, a receptor of a target of the present invention, and a downstream effector, or regulators thereof. The incorporation of a desired polynucleotide into a vector and the choice of vectors is well-known in the art as described in, for example, Sambrook et al., supra, and Ausubel et al., supra.

The siRNA polynucleotide can be cloned into a number of types of vectors. However, the present invention should not be construed to be limited to any particular vector. Instead, the present invention should be construed to encompass a wide plethora of vectors which are readily available and/or well-known in the art. For example, an siRNA polynucleotide of the invention can be cloned into a vector including, but not limited to a plasmid, a phagemid, a phage derivative, an animal viruse, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient endonuclease sites, and one or more selectable markers. (See, e.g., WO 01/96584; WO 01/29058; and U.S. Pat. No. 6,326,193.

For expression of the siRNA, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 by apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of Inhibiting a Gene of a Target of the Present Invention and mRNA Expression

### Antisense Nucleic Acids

In one embodiment of the invention, an antisense nucleic acid sequence which is expressed by a plasmid vector is used to inhibit a target of the present invention, a receptor of a target of the present invention, or a downstream effector expression of a target of the present invention. The antisense expressing vector is used to transfect a mammalian cell or the mammal itself, thereby causing reduced endogenous expression of a target of the present invention, a receptor of a target of the present invention, or a downstream effector expression of a target of the present invention. Examples of downstream effectors include, but are not limited to, extracellular matrix proteins (collagens, fibronectin, elastin, fibrillin), CCN proteins 1-5 (Cyr61, connective tissue growth factor, NOV3, Wisp-1, Wisp-2), fibroblast growth factor-2, IL-13 receptor components, proteases (cathespsins-S, -K, -B, and -H), antiproteases, and apoptosis regulators (Bax, TNF, and early response gene-1).

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

### Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit a target of the present invention, a receptor of a target of the present invention, or a downstream effector expression of a target of the present invention. Ribozymes useful for inhibiting the expression of a target molecule may be designed by incorporating target sequences into the basic ribozyme structure which are complementary, for example, to the mRNA sequence of a target of the present invention. Ribozymes targeting a target of the present invention, a receptor of a target of a present invention, or a downstream effector thereof, may be synthesized using commercially available reagents (Applied Biosystems, Inc., Foster City, Calif.) or they may be genetically expressed from DNA encoding them.

### Assays for Identifying and Testing Candidate Inhibitors of a Target of the Present Invention

Inhibitors of gene expression, mRNA stability and expression, and protein activity, function and expression of a target of the present invention, a receptor of a target of the present invention, and downstream effectors can be identified by screening test compounds. For instance, inhibitors of gene expression of an endogenous target of the present invention or of the expression of a target of the present invention can be identified by screening test compounds for their capacity to reduce or preclude the gene expression of a target of the present invention or mRNA expression of a target of the present invention in a cell, preferably a pulmonary endothelial cell. The coding sequence of a target of the present invention in such screening assays may include an in-frame fusion of a tag to the coding sequence of a target of the present invention. Such tags enable monitoring of expression of a target of the present invention by antibody detection of the tags or spectral methods of detection (e.g., fluorescence or luminescence).

Test compounds for use in such screening methods can be small molecules, nucleic acids including aptamers, peptides, peptidomimetics and other drugs. Peptide fragments of a target of the present invention are contemplated that can competitively inhibit the binding of a target of the present invention to a cognate receptor, thereby inhibiting activity of a target of the present invention. Peptide fragments of a target of the present invention that include the known Arg-Gly-Asp (RGD). β1 integrin binding domain are preferred in the present invention.

The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145) Inhibitors and activators expression of a target of the present invention may be useful in therapeutic applications, or serve as lead drugs in the development of therapeutics. Synthetic techniques may be used to produce compounds, such as: chemical and enzymatic production of small molecules, peptides, nucleic acids, antibodies, and other therapeutic compositions useful in the practice of the methods of the invention. Other techniques may be used which are not described herein, but are known to those of skill in the art.

In one aspect of the invention, libraries of small molecules, including but not limited to aptamers, peptidomimetics, peptide fragments of a target of the present invention, or peptidomimetics, may be assayed for competitive binding to one or both known Sema 7a receptors, a target of the present invention, or any receptor of a target of the present invention identified in the future. In another aspect of the invention, TGF-β1 induction in the presence of a target of the present invention of at least one relevant downstream gene is assayed in the presence and absence of a test compound. Relevant downstream genes include: collagens (e.g., α1(I), α1(II) and type III collagens, fibronectin (FN), elastin, laminin); protease (e.g., TIMP-1); fibroregulatory cytokines and their receptors (e.g., CCN1 (Cyr-61), CCN2 (connective tissue growth factor; CTGF), CCN3 (NOV), CCN4 (WISP-1) and CCN5 (WISP-2) and the IL-13 and IL-4 receptor components IL-4Rα and IL-13Rα2). IL-13 mediates its fibrogenic effects, in part, via its ability to activate TGF-β1. It has been demonstrated that inhibiting Sema 7A limits IL-13 induced fibrogenesis (Elias et al., U.S. patent application Ser. No. 12/604,192, hereby incorporated by reference in its entirety). In yet another aspect of the invention, changes in IL-18 expression are assayed in response to administration of a test compound. IL-18 is known to inhibit tissue fibrosis, but as previously demonstrated, Sema 7a inhibits IL-18 production (Elias et al., U.S. patent application Ser. No. 12/604,192, hereby incorporated by reference in its entirety). In still another aspect of the invention, an in vivo assay is performed to assay a stabilization and reduction of alveolar remodeling as a result of administering an inhibitor of a target of the present invention.

In another embodiment of the invention, an in vitro binding assay is used to determine binding affinity and dissociation kinetics of potential inhibitors of a target of the present invention for a target of the present invention, receptors of a target of the present invention, and downstream effectors of a target of the present invention. Examples of in vitro binding assays are well known in the art. Standards may be used when testing new agents or compounds or when measuring the various parameters described herein. For example, TGF-β1 can be administered to a group or subject as a standard or control against which the effects of a test agent or compound can be compared. In addition, when measuring a parameter, measurement of a standard can include measuring parameters such as a target of the present invention or concentrations of a target of the present invention in a tissue or fluid obtained from a subject before the subject is treated with a test compound and the same parameters can be measured after treatment with the test compound. In another aspect of the invention, a standard can be an exogenously added standard which is an agent or compound that is added to a sample and is useful as an internal control, especially where a sample is processed through several steps or procedures and the amount of recovery of a marker of interest at each step must be determined. Such exogenously added internal standards are often added in a labeled form, i.e., a radioactive isotope.

Inhibitors of a target of the present invention useful in the invention may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the .alpha -amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the .alpha.-amino protecting group, and the FMOC method, which utilizes 9-fluorenylmethyloxcarbonyl to protect the .alpha.-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a SEMA 7A inhibitor in accordance with the invention, a peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### Agonists/Activators

In another embodiment, the invention provides compositions and methods for activating Plexin C1 in order to inhibiting fibrosis in a mammal Agents which are capable of increasing Plexin C1 levels and/or activity are referred to as agonists or activators of that activity.

The term “agonist”, as used in the art, is generally taken to refer to a compound which binds to an enzyme and promotes the activity of the enzyme. The term as used here, however, is intended to refer broadly to any agent which enhances or promotes the activity of a molecule, not necessarily by binding to it. Accordingly, as used generally, it includes agents which affect activity of Plexin C1.

The agonist or activator may promote the binding of the relevant molecule, for example, Plexin C1, to an upstream protein.

Enhancing the activity of Plexin C1 may also be achieved by increasing the level of expression of Plexin C1 in the cell. For example, the cell may be treated with nucleic acid sequences encoding Plexin C1.

As used herein, in general, the term “agonist” includes but is not limited to agents such as an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex.

The terms “agonist” and “agent” are also intended to include, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanized, a peptide hormone, a receptor, a signaling molecule or other protein; a nucleic acid, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

### Pharmaceutical Compositions and Therapies

Administration of an inhibitor of a target of the present invention comprising one or more peptides, small molecules, antisense nucleic acids, soluble receptors, or antibodies of the invention in a method of treatment can be achieved in a number of different ways, using methods known in the art. Such methods include, but are not limited to, providing exogenous peptide inhibitor, small molecule, soluble receptor, or an antibody to a subject or expressing a recombinant peptide inhibitor, small molecule, soluble receptor, or an antibody expression cassette.

In one embodiment, an exogenous SEMA 7A inhibitor peptide is administered to a subject. The exogenous peptide may also be a hybrid or fusion protein to facilitate, for instance, delivery to target cells or efficacy. In one embodiment, a hybrid protein may comprise a tissue-specific targeting sequence.

In another embodiment, an expression vector comprising an expression cassette encoding an inhibitor protein of a target of the present invention, or fragment thereof, or an antibody that will bind an epitope specific to a target of the present invention, a soluble SEMA 7A receptor, or a fragment thereof, is administered to a subject. An expression cassette may comprise a constitutive or inducible promoter. Such promoters are well known in the art, as are means for genetic modification. Expression vectors and methods for the introduction of exogenous DNA into cells with concomitant expression of the exogenous DNA in the cells are described, for example, in Sambrook et al. (2001, Molecular Cloning: A Laboratory Manual, Cold Spring Harbor Laboratory Press, Cold Spring Harbor, N.Y.), and in Ausubel et al. (eds, 2005, Current Protocols in Molecular Biology, John Wiley & Sons, New York, N.Y.). In one embodiment, a cell comprising an expression vector of the invention is administered to a subject. Thus, the invention encompasses a cell comprising an isolated nucleic acid encoding an inhibitory peptide of a target of the present invention, fusion protein or antibody of the invention.

Any expression vector compatible with the expression of an inhibitory peptide of a target of the present invention, fusion protein, soluble receptor, or antibody of the invention and for a target of the present invention is suitable for use in the instant invention, and can be selected from the group consisting of a plasmid DNA, a viral vector, and a mammalian vector. The expression vector, or a vector that is co-introduced with the expression vector, can further comprise a marker gene. Marker genes are useful, for instance, to monitor transfection efficiencies. Marker genes include: genes for selectable markers, including but not limited to, G418, hygromycin, and methotrexate, and genes for detectable markers, including, but not limited to, luciferase and GFP. The expression vector can further comprise an integration signal sequence which facilitates integration of the isolated polynucleotide into the genome of a target cell.

The therapeutic and prophylactic methods of the invention thus encompass the use of pharmaceutical compositions comprising an inhibitory peptide, fusion protein, small molecule, soluble receptor, or antibody of a target of the present invention and/or an isolated nucleic acid encoding an inhibitory peptide, fusion protein small molecule, soluble receptor, or antibody of a target of the invention to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg is to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising an inhibitor of a target of the invention and an instructional material which describes, for instance, administering the inhibitor of a target of the invention to a subject as a prophylactic or therapeutic treatment or a non-treatment use as described elsewhere herein. In an embodiment, this kit further comprises a (preferably sterile) pharmaceutically acceptable carrier suitable for dissolving or suspending the therapeutic composition, comprising an inhibitor of a target of the invention, for instance, prior to administering the molecule to a subject. Optionally, the kit comprises an applicator for administering the inhibitor. In one embodiment of the invention, the applicator is designed for pulmonary administration of the inhibitor of a target of the invention. In another embodiment, the kit comprises an antibody that specifically binds an epitope on a target of the present invention. Preferably, the antibody recognizes a human target of the present invention.

A kit providing a nucleic acid encoding a peptide or antibody of the invention and an instructional material is also provided.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Semaphorin 7a-Induced Lung Fibrogenesis Occurs in a CD4-Dependent, Macrophage-Dependent Manner

The results described herein show that Sema 7a is a robust inducer of M2 activation in both the mouse and human and define a new role for CD4+ T cells in these responses. The results indicate that Sema 7a and related signaling partners are increased in the lung and/or blood of IPF patients. Sema 7a is elevated on CD19+ and CD4+ lymphocytes compared to non-fibrotic controls. Disease progression is predicted both by Sema 7a expressing CD4+ cells and expression of CD206 on circulating monocytes. Murine studies indicate that Sema 7a-expressing CD4+ cells are sufficient, but not required, for M2 accumulation and fibrosis in a mouse model of pulmonary fibrosis caused by doxycycline inducible, lung specific overexpression of TGF-β1. These effects are accompanied by increased levels of the actin-modifying protein Cofilin-1. CD206 expression is greatly increased when Sema 7a's cognate receptor, Plexin C1 (a Cofilin inactivator), is inhibited. These results are consistent with the theory that targeting Sema 7a, CD4 cells, and/or Cofilin-1 may be of therapeutic benefit in TGF-β1-associated, macrophage-driven fibrotic disorders such IPF.

The materials and methods employed in these experiments are now described.

### Materials and Methods

Transgenic Mice

All mouse experiments were approved by the Yale School of Medicine Institutional Animal Care and Use Committee. The CC10-tTS-rtTA-TGF-β1 transgenic mice used in this study have been described previously 10. These mice use the Clara cell 10-kDa protein (CC10) promoter to specifically express bioactive human TGF-β1 to the lung, The Semaphorin 7a null mice have been described previously (Pasterkamp et al., 2003, Nature 424:398-405) and were a gift from Dr. Alex Kolodkin, Johns Hopkins University. The CD4tmlMak and CD19tml(CRE)cgn mice were obtained from Jackson Laboratories, Bar Harbor, Me. All mice were backcrossed for >10 generations onto a C57BL/6 background.

Human Subjects

These studies were performed with approval from the Human Investigation Committee at the Yale School of Medicine. Patients were recruited from the Interstitial Lung Disease program at Yale University School of Medicine. All patients with a diagnosis of IPF based on current ATS criteria (Am. J. Respir. Crit. Care. Med. 165:277-304) were eligible to enroll. Patients were excluded based on the following criteria: (Homer et al., 2011, Arch. Pathol. Lab. Med. 135:780-788) Inability to provide informed consent; (Gen et al, Ther. Clin. Risk. Manag. 7:39-47) Significant non-vascular lung disease; (Noble et al., 2004, Clin. Chest Med. 25:749-758) Unstable cardiac, vascular, or neurologic disease in past 6 months; (Jiang et al., 2005, Nat. Med. 11:1173-1179) Malignancy; (Murray et al., 2010, PLoS One 5:e9683) Pregnancy; (Murray et al., 2011, Int. J. Biochem. Cell Biol. 43:154-162) Chronic infection. Comprehensive clinical data including age, sex, race and ethnicity, co-morbidities, medications and physiologic impairment as measured by the percent-predicted Forced Vital Capacity (FVC) and Diffusion Capacity of Monoxide (DLCO) were collected. A group of normal age-matched controls was recruited from the local community and from the Yale Program on Aging.

Doxycycline Administration

TGF-β1 Tg+ or their wild-type littermate controls (transgene negative, Tg−) mice were given doxycycline 0.5 mg/ml in their drinking water from aged 8-10 weeks up to 2 weeks.

Bone Marrow Transplantation

Bone marrow chimeras of the TGF-β1 and Sema 7a−/− mice were created as previously described (Gan et al., 2011, Arthritis Rgeum. 63:2484-2494).

BAL and Sacrifice

Euthanasia, bronchoalveolar lavage, and tissue harvest were performed as previously described (Gan et al., 2011, Arthritis Rgeum. 63:2484-2494).

Processing of Lungs for Flow Cytometry

Lungs were digested for flow cytometry and total viable cells were quantified using Trypan blue staining as previously described (Gan et al., 2011, Arthritis Rgeum. 63:2484-2494).

Flow Cytometric Analysis

Antibodies against mouse F4/80, CD11b, and CD11c, and against human CD4, CD8, and CD14 were purchased from BD Pharmingen. CD206 antibody was purchased from Serotec. Semaphorin 7a antibody was obtained from R & D Systems. Appropriate isotype controls were obtained from BD Pharmingen. Flow cytometry and cell sorting were performed using a BD FACSCalibur. Data were analyzed using Flow Jo v 7.5 software (Tree Star, Inc, Ashland, Oreg.). For all analyses, isotype control staining was subtracted from true antibody staining to determine the percentage of positive cells. When necessary this percentage was then multiplied by the total cell count in the sample to derive absolute quantity of the cell population of interest.

Clodronate Depletion of Intrapulmonary Macrophages

Macrophages in the lung were depleted in TGFβ-Tg+ or Tg− mice intranasally delivered liposomal clodronate following doxycycline initiation. It was encapsulated in liposomes as described earlier (Van Rooijen et al., 1994, J. Immunol. Methods 174:83-93).

Sircol Analysis

Lung collagen was measured using the Sircol Assay following manufacturer's protocol (Biocolor Ltd., UK).

Histologic Analysis

Formalin-fixed and paraffin-embedded lung sections were stained with hematoxylin and eosin to assess gross morphology or Mallory's trichrome stains to visualize collagen deposition.

Immunofluorescence

Immunofluoresence of digested mouse lung cytospins was performed using primary antibodies against CD19, CD4 (both from BD Pharmingen), Sema 7a (Genetex, Irvine Calif.), and CFL-1 (Abcam, Boston, Mass.). Secondary antibodies were purchased from Invitrogen.

Processing Of Human Blood for Flow Cytometry

Ficoll separation and processing of PBMCs for FACS was performed as we have previously described (Gan et al., 2011, Arthritis Rgeum. 63:2484-2494).

Quantitative Real Time PCR RNA was isolated from samples and reverse transcribed according to our previous methods (Murray et al., 2011, Int. J. Biochem. Cell Biol. 43:154-162). Primers against mouse MRC, MSR1, CXCL10, and CFL1, and against human Sema 7a, Plexin C1, CFL-1 were obtained from Superarray Bioscience (Frederick, Md.).

Sema 7a Stimulation of Monocytes

CD14+ monocytes were obtained via MACS-enrichment as have been previously described. These cells were stimulated with recombinant Sema 7a (Abnova, Taiwan) or human serum albumin (Sigma) as previously described (Gan et al., 2011, Arthritis Rgeum. 63:2484-2494). Lentivirally-mediated shRNA knockdown of Plexin C1 expression was performed using specific anti-Plexin shRNA or control “scramble” shRNA (Santa Cruz Biotech, Santa Cruz Va.) as previously described (Gan et al., 2011, Arthritis Rgeum. 63:2484-2494).

Statistical Analysis

Normally distributed data are expressed as mean±S.E.M. and assessed for significance by Student's t-test or ANOVA as appropriate. Data that were not normally distributed were assessed for significance using the Mann-Whitney U test where appropriate. Statistical analysis was performed using SAS biostatistical software (Research Triangle Park, N.C.) and graphs were generated using Graphpad (Graphpad Software Inc., La Jolla, Calif.).

The results of the experiments are now described.

### Semaphorin 7a Promotes Alternative Macrophage Activation in the TGF-β1 Exposed Murine Lung

In order to determine whether Sema 7a is involved in M2 macrophage activation, a well-characterized murine model of macrophage-driven pulmonary fibrosis was used in which the bioactive form of the human TGF-β1 gene is placed under control of a doxycycline inducible, lung specific promoter (Lee et al., 2004, J. Exp. Med. 200:377-389). In the setting of doxycyline administration, these mice develop an epithelial cell death response that is followed by the influx of CD206+ M2 macrophages and, eventually, the development of collagen accumulation and fibrosis (Murray et al., 2011, Int. J. Biochem. Cell Biol. 43:154-162; Lee et al., 2004, J. Exp. Med. 200:377-389; Gan et al., 2011, Arthritis Rheum. 63:2484-2494). These mice are referred herein as “TGF-β1 mice.” In order to determine whether Sema 7a plays a role in the M2 activation seen in this model, the TGF-β1 transgene positive (Tg+) mice and their wild-type littermates (Transgene-negative, Tg−) were crossed with mice carrying null mutations of the Sema 7a locus (Sema 7a−/−) and the effect on TGF-β1-relevant endpoints such as lung inflammation (measured by BAL cell counts), lung fibrosis (assessed histologically and quantified by Sircol analysis) and M2 activation (assessed by qRT-PCR for M2-associated genes and by flow cytometry for CD206+ lung macrophages) were assessed.

Sema 7a deletion was found to have no effect on lung inflammation following 14 days of TGF-β1 over-expression (FIG. 1A), but led to a significant reduction in the accumulation of collagen in the lung (p<0.0001, FIG. 1B) and improvement in the histologic appearance of fibrosis (FIG. 1C), compared to TGFβ Tg+ control mice. These effects were accompanied by a 72.2% decrease in the expression of MRC/CD206 (p<0.05) and a 76.9% reduction in the expression of MSR1 (p<0.05, FIG. 1D) in whole lung RNA, and by a 59.6% reduction in quantities of lung macrophages expressing the M2 marker CD206 (p<0.05, FIG. 1E), as measured by flow cytometry. These results indicate that in the setting of Sema 7a deletion, M2 macrophages are also reduced, and although not wishing to be bound by any particular theory, also suggests this relationship might be important in the development of fibrosis.

### Sema 7a Expression is Increased in the Lungs of IPF Patients

Having found a relationship between Sema 7a and M2 activation in a murine model of pulmonary fibrosis, it was examined whether there also exists a correlation with human disease. Idiopathic pulmonary fibrosis is a fatal form of lung fibrosis that is associated with high levels of TGF-β1 expression and enhanced M2 activation in both the lungs and the blood (Murray et al., 2011, Int. J. Biochem. Cell Biol. 43:154-162; Murray et al., 2010, PLoS One 5:e9683). Whether high levels of Sema 7a are also seen in lungs from these patients was examined. Tissue was obtained from the Lung Tissue Research Consortium (LTRC) or from the tumor free margin of cancer resections performed at Yale and the expression of Sema 7a and was assessed at the message level by q-RT-PCR and at the protein level by immunohistochemistry. All samples were assessed by a lung pathologist to confirm the presence of UIP pathology and the absence of cancer. As shown in FIG. 2, compared to histologically normal lung in which the relative expression of Sema 7a was quite low, lungs of patients with IPF demonstrated 2.42-fold increased expression of Sema 7a (p=0.027, FIG. 2A). This increased expression was confirmed at the protein level where immunohistochemical analysis revealed that, in contrast to normal lung, in which Sema 7a was nearly undetectable, IPF lung contains a more than 20-fold robust increase in Sema 7a-expressing cells (p<0.0001, FIG. 2B). The majority of Sema 7a expression was seen adjacent to fibroblastic foci in cells possessing the morphologic characteristics of lymphocytes. The extreme autofluorescence of the IPF lungs made further identification of these cells by double labeling impossible. Little to no expression was seen in epithelial cells and fibroblasts (FIGS. 2C, 2D, and 8). These data indicate that Sema 7a expression is increased in the IPF lung and that much of this increase is accounted for by the presence of Sema 7a+ hematopoietic cells.

Sema 7a Expression is Increased in the Blood of IPF Patients where it Localizes to CD4 and CD19 Cells

In view of the fact that Sema 7a was located primarily on hematopoietic cells in the IPF lungs, experiments were designed to determine whether Sema 7a could be detected in the blood of these patients. Sema 7a in RNA obtained from the peripheral blood mononuclear cells (PBMCs) of normal subjects (n=34) was compared with samples from subjects with IPF (n=23). Patient characteristics are shown in Table 1. Similar to the lung, the blood of IPF patients contained a 3.49-fold increase in the relative expression of Sema 7a mRNA (p=0.0025, FIG. 3A).

TABLE 1
Demographic Characteristics of Subjects

| | Control | | IPF | | P value |
|---|---|---|---|---|---|
| Age, years | 73.65 | (71.10-76.20) | 69.16 | (65.52-72.79) | 0.037 |
| Sex | | | | | |
| Female | 17 | (50.00%) | 3 | (13.04%) | 0.004 |
| Male | 17 | (50.00%) | 20 | (86.96%) | |
| Race | | | | | |
| Caucasian | 32 | (94.12%) | 19 | (82.60%) | 0.021 |
| Other | 2 | (5.88%) | 4 | (17.40%) | |

| | | | |
|---|---|---|---|
| FVC, | N/A | 63.67 +/− 3.58 | N/A |
| percent pred. | | | |
| DLCO, | N/A | 44.30 +/− 4.93 | N/A |
| percent adj. | | | |

The cell population or populations responsible for this increased expression were next determined. Because total post-ficoll cell counts did not differ between control and IPF subjects, differences in the percentages of specific leukocyte subpopulations that express Sema 7a were examined. If Sema 7a were involved in M2 activation, it was examined whether it would either be expressed on monocytes or expressed by cells that modulate monocyte differentiation such as lymphocytes. Although an increase in Sema 7a+CD14+ monocytes between control and IPF samples (p=0.61, FIG. 9) was not found, it was examined whether the increase in Sema 7a expression could be explained by augmented expression on lymphocytes. This was found to be correct as, in comparison to control values, the percentage of CD19+ lymphocytes co-expressing Sema 7a was increased by 48.7% in this group (p=0.04, FIGS. 3B and 3C) and the percentage of CD4+ cells expressing Sema 7a was increased by 64.5% (p=0.02, FIGS. 3D and 3E). In contrast, Sema 7a+ CD8+ cells did not differ between groups (p=0.18, FIG. 9), indicating that the increased detection of Sema 7a in the blood of IPF patients is due in a large part to enhanced expression on CD4+ cells and CD19+ cells.

### Sema 7a Expression on Circulating CD4+ Cells, but not CD19+ Cells, is Increased in Rapidly Progressive IPF

The biologic relevance of these Sema 7a expressing lymphocytes was examined. The IPF cohort was followed prospectively for 9 months and stratified into “stable” or “progressive” based on the following composite outcomes: >10% decline in FVC, acute exacerbation, or death. Clinical characteristics of these patients are shown in Table 2.

TABLE 2
Demographic Characteristics of Progressive vs.
Stable IPF Patients

| | Progressive | | Stable | | |
|---|---|---|---|---|---|
| | N = 12 | | N = 11 | | P value |
| Age, years | 67.39 | (61.85-72.93) | 71.09 | (65.71-76.47) | 0.302 |
| Sex | | | | | |
| Female | 0 | (0%) | 3 | (27.27%) | 0.052 |
| Male | 12 | (100.00%) | 8 | (72.73%) | |
| Race | | | | | |
| Caucasian | 9 | (75.00%) | 9 | (90.00%) | 0.364 |
| Other | 3 | (25.00%) | 2 | (10.00%) | |
| Smoking | | | | | |
| Ever | 11 | (91.67%) | 7 | (63.66%) | 0.08 |
| Never | 1 | (8.33%) | 4 | (37.44%) | |
| Diagnosis | | | | | |
| based on | | | | | |
| HRCT | 3 | (25.00%) | 4 | (36.36%) | 0.496 |
| Pathology | 9 | (75.00%) | 7 | (73.64%) | |
| FVC, percent | 63.45 | (55.20-71.71) | 63.89 | (49.75-78.02) | 0.954 |
| predicted % | | | | | |
| DLCO %, | 38.82 | (27.47-50.17) | 46.72 | (32.56-60.87) | 0.344 |
| percent | | | | | |
| predicted | | | | | |

The levels of Sema 7a+ lymphocytes in the stable subjects were compared with those of progressive subjects. It was found that in contrast to those IPF patients demonstrating short-term clinical stability, IPF patients who went on to experience accelerated clinical decline showed greater than seven-fold increase in quantities of Sema 7a-expressing CD4+ cells (p=0.03, FIG. 3F). There was no appreciable difference between Sema 7a+ CD19+ cells in these subjects (p=0.32, FIG. 3F). Interestingly, rapidly progressive patients also showed a 2.07-fold increase in levels of circulating of CD206+ monocytes (p=0.0017, FIGS. 3G and 10), suggesting a link between Sema 7a+CD4+ cells, M2 activation, and the progression of fibrotic lung disease.

### Hematopoietic Expression of Sema 7a is Sufficient for M2 Accumulation and Fibrosis in the TGF-β1 Exposed Murine Lung

Murine data had shown that Sema 7a importantly regulates the alternative activation of macrophages in TGF-β1 induced lung fibrosis and the human model has shown that Sema 7a+ cells are increased in the blood and lungs of patients with IPF. However, whether the biological relevance of hematopoietic expression of Sema 7a exerts these effects remained unclear. Bone marrow chimeras were synthesized to determine whether Sema 7a expressing bone marrow derived cells (BMDCs) are necessary, sufficient, or both for the induction of M2 activation in the TGF-β1 exposed murine lung. It was observed that in chimeras in which Sema 7a had been removed from the bone marrow, BAL cell counts were largely unchanged (p=0.88, FIG. 4A) and there were only modest, and not significant, reductions in TGF-β1 induced lung fibrosis (p=0.13, FIG. 4B) and M2 accumulation (p=0.11, FIG. 4C). In contrast, in the TGF-β1×Sema 7a−/− mice in which Sema 7a had been restored on BMDCs, BAL cell counts were also unchanged (p=0.35, FIG. 4A) but collagen accumulation was increased by 2.3-fold percent (p=0.001, FIGS. 4B and 4C) and M2 content was increased by 3.64-fold percent (p=0.003, FIG. 4D), both of which approximated levels in the TGF-β1×Sema 7a+/+ animals (FIG. 4C). These data indicate that Sema 7a expressing BMDCs are sufficient for the appearance of M2 macrophages and fibrosis in the TGF-β1 exposed murine lung.

Sema 7a-Induced Lung Fibrosis is Reduced by Macrophage Depletion

Previous studies demonstrated that removal or modulation of macrophage phenotypes attenuates collagen accumulation in experimentally induced lung fibrosis (Murray et al., 2011, Int. J. Biochem. Cell Biol. 43:154-162; Murray et al., 2010, PLoS One 5:e9683). However, it was unclear as to whether the fibrosis induced by the transfer of Sema 7a BMDCs was also macrophage dependent. Liposomal clodronate was used to deplete macrophages in the chimeric mice in which Sema 7a was restricted to the circulation (TGF-β1×Sema 7a−/− recipients of Sema 7a+/+ bone marrow). This approach led to a 27.1% reduction in collagen accumulation (p=0.021, FIG. 4E) indicating that the pro-fibrotic effects of Sema 7a occur, at least in part, via a macrophage dependent mechanism.

### Sema 7a is Expressed Robustly on CD4+ Cells, but not CD19+ Cells, in the TGF-β1 Exposed Murine Lung

The data presented herein indicated that Sema 7a induced M2 activation in experimentally induced lung fibrosis in mice, that Sema 7a was increased in CD4+ and CD19+ cells in the blood of patients with IPF, and that replacement of Sema 7a on BMDCs was sufficient for M2 accumulation and fibrosis in the TGF-β1 exposed murine lung. Without being bound by any particular theory, it is believed that these effects can be related to Sema 7a expression on CD4+ or CD19+ cells, and therefore Sema 7a expression was assessed on these cells in the model. Immunofluorescence by way of FACS analysis was performed on cells obtained from the lungs of TGF-β1 mice for the presence of Sema 7a and either CD4 or CD19. It was observed that while nearly all of the CD4+ cells also co-expressed Sema 7a (FIG. 5A), there was minimal co-expression of Sema 7a and CD19 (FIG. 5B), consistent with the theory that CD4+ cells may participate in Sema 7a-induced fibrosis.

### Sema 7a Expression by CD4, but not CD19 Cells, Promotes M2 Accumulation and Fibrosis in the TGF-β1 Exposed Murine Lung

Human data indicated that the increased Sema 7a expression seen in IPF patients stems largely from increased expression on two lymphocyte populations. In these samples, CD4 lymphocytes demonstrated a biologic relationship with M2 activation and disease progression while CD19 lymphocytes did not. In the mouse lung it was observed that Sema 7a was highly expressed on CD4+ and not CD19+ cells. In order to determine whether either of these populations mediate the M2 effects and fibrosis seen in the model, the chimeric mice in which Sema 7a was restricted to the circulation were used. However, donors with null mutations of either CD4 or CD19 were also used. Following the transplantation and recovery period, mice were given doxycycline for 14 days and assessed for M2 content by flow cytometry and for fibrosis by Sircol and histology. As expected, TGF-β1×Sema 7a−/− mice transplanted with CD4 null BDMCs did not develop increased BAL inflammation (p=0.22, FIG. 5C), M2 accumulation (p=0.02, FIG. 5D) and fibrosis (p=0.02, FIGS. 5E and 5F) seen in the recipients of Sema 7a+/+ WT marrow. In contrast, TGF-β1×Sema 7a−/− transplanted with CD19 null marrow showed sustained levels of lung inflammation, M2 macrophages, and collagen accumulation (p>0.05 all comparisons, FIGS. 5C-5F). Importantly, genes for M1 activation such as CXCL10 were increased by 2.23-fold in the recipients of CD4 null marrow (p=0.03, FIG. 5G), suggesting that the CD4 effects in this model are specific to M2 macrophages and not abnormalities in macrophage activation in general. These data indicate that CD4 lymphocytes are required for Sema 7a-expressing BMDC to induce TGF-β1 induced M2 accumulation and fibrosis.

### Sema 7a Stimulation is Sufficient for M2 Activation of Normal, but not IPF, Monocytes

These data indicated that Sema 7a expression on BMDCs is sufficient for the development of M2 responses and fibrosis in a murine model of experimentally induced lung disease. However, the importance of this pathway in human responses has not yet been defined. Thus, monocytes from normal controls and IPF subjects were isolated and cultured in the presence or absence of recombinant Sema 7a protein, after which they were harvested and assessed for CD206 expression via flow cytometry. Somewhat unexpectedly, while normal human monocytes responded to Sema 7a stimulation by displaying a 94.3% increase in cell surface expression of CD206 (p=0.04, FIGS. 6A and 6B), IPF monocytes, which already displayed markedly increased cell surface expression of this protein, failed to respond to Sema 7a stimulation (p=0.16, FIGS. 6A and 6B).

### Increased Expression of Plexin C1 and Cofilin-1 in IPF

These data led to a reexamination of whether Sema 7a drives M2 activation and fibrosis in IPF and several alternatives were considered: either Sema 7a does not promote M2 activation in IPF; the M2 capacity is fully reached and cannot be exceeded; or that the monocytes of IPF patients express a factor that is inhibitory for CD206 expression. Given the robust animal data described elsewhere herein, the first two possibilities were considered to be unlikely. The third possibility regarding an inherent inhibitor to Sema 7a was examined.

Sema 7a signals through two known receptors: the β1 integrin subunit and Plexin C1. While the role of β1 integrin in Semaphorin biology and fibrosis has been extensively studied, the role of Plexin C1 is less well understood. Plexin C1 is the cognate receptor for Sema 7a through which it exerts inhibitory effects on cytoskeletal rearrangement via inactivation of the actin-modifying protein Cofilin-1 (CFL1) (Lazova et al., 2009, Am. J. Dermatopathol. 31:177-181). CFL1 is known to be one of the most highly expressed proteins when human alveolar macrophages are compared to circulating monocytes (Jin et al., 2004, Am. J. Respir. Cell Mol. Biol. 31:322-329). To date, however, this pathway has not been explored in either M2 activation or IPF.

In order to determine whether the Plexin C1-CFL interaction may be attenuating Sema 7a-induced CD206 expression in the IPF subjects, expression of these genes in the blood and lungs of patients with IPF was assessed. It was observed that in contrast to normal controls, both Plexin C1 and CFL1 were significantly increased in monocytes (p<0.05 each comparison, FIGS. 6C and 6D), but not whole lung tissue, from the blood of IPF patients (FIGS. 6C and 6D). Although not wishing to be bound by any particular theory, these results indicated a possible role for these proteins in the regulation of the monocyte phenotypes. Next, a functional relationship between these genes was examined by assessing the effect of lentiviral-mediated Plexin C1 knockdown on Sema 7a-induced CD206 activation. It was observed that inhibition of Plexin C1 expression allowed a 23.3% increase in M2 activation in the IPF samples (p=0.0017, FIG. 6E) but had no effect on control cells (p=0.834, FIG. 6 E). These data indicate that Sema 7a-induced M2 activation is opposed by Plexin C1, and although not wishing to be bound by any particular theory, these data support a role for CFL1 in this process.

### CFL-1 is Increased in the TGF-β1 Exposed Murine Lung

In order to determine whether CFL1 is also involved in Sema 7a's effects on TGF-β1 induced M2 activation and lung fibrosis, expression of CFL1 in the mouse model was assessed using immunofluorescence. High level expression of CFL1 was observed in a variety of cells including F4/80+ lung macrophages (FIG. 7A), thereby indicating a role for this pathway in the model.

CFL1 is Implicated in Sema 7a-Induced M2 Activation and Fibrogenesis

In order to determine whether CFL1 was associated with Sema 7a-induced M2 activation and fibrosis in the mouse, CFL1 expression was assessed in the lungs of the recipients of Sema 7a+BMDCs. It was observed that CFL1 expression was high in TGF-β1×Sema 7a+/+ mice that received WT marrow and that CFL1 expression was not affected in those TGF-β1 mice in which Sema 7a had been removed from BMDCs via transplantation. In contrast, the TGF-β1×Sema 7a−/− animals that received Sema 7a−/− marrow showed low level expression of CFL1 that was increased by 60.1% by replacement of Sema 7a via bone marrow transplantation (p=0.0003, FIG. 7B). These data indicate that Sema 7a's induction of lung fibrosis by hematopoietic cells involves increased expression of Cofilin-1, thus further supporting a role for this protein in the M2 activation and fibrogenesis caused by transfer of Sema 7a+BMDCs.

Sema 7a, CD4 Cells, M2 Activation, and CFL1 May Represent a Novel Therapeutic Target in Patients with IPF

Alternatively activated macrophages have been implicated as causative in diverse fibrotic diseases. Semaphorin 7a is a novel regulator of both monocyte cell fate and fibrosis in the TGF-β1 exposed murine lung. It was examined whether Sema 7a signaling is increased in Idiopathic Pulmonary Fibrosis (IPF) patients and exerts its effects on lung fibrosis in part through effects on CD206+ alternatively activated macrophages (“M2”). It was also examined whether semaphorin 7a exerts important effects on M2 activation and fibrosis in the setting of idiopathic pulmonary fibrosis, while the role of lymphocytes in these processes was also explored.

These studies lend new insight into the immunologic mechanisms driving M2 activation and fibrosis in the TGF-β1 exposed murine lung and in primary cells obtained from patients with IPF. Specifically, they demonstrate a novel relationship between Sema 7a expressing CD4+ cells, M2 macrophages, and disease progression in patients with IPF. Sema 7a induces M2 activation in normal human monocytes and is found with increased frequency on both CD19+ lymphocytes, and CD4+ lymphocytes in the IPF patients; however, only Sema 7a+CD4+ cells were increased in those patients who show an accelerated clinical course and enhanced M2 activation. Although not wishing to be bound by any particular theory, these data suggest that Sema 7a+ CD4+ cells may not only function as a biomarker of disease activity but may also participate in M2 activation and fibrogenesis in the setting of fibrotic lung disease. These data also indicate that Sema 7a+ hematopoietic cells are sufficient for the development of fibrosis in the TGF-β1 exposed lung and that these effects are mediated via CD4+ lymphocytes that induce M2 activation of macrophages and increased expression of CFL1. This axis is also present in human with IPF, where Sema 7a stimulates alternative macrophage activation in a manner that is opposed by Plexin C1's inhibitory phosphorylation of Cofilin-1 (CFL1).

Alternative macrophage activation has emerged as an important driver of TGF-β1 associated fibrosis in diverse clinical settings including but not limited to IPF (Murray et al., 2011, Int. J. Biochem. Cell Biol. 43:154-162), scleroderma (Mathai et al., 2010, Lab. Invest. 90:812-823), and renal fibrosis (Castano et al., 2009, Sci. Transl. Med. 1:5ra13). While the precise mechanism(s) through which these cells promote fibrosis remain unknown, prior work has shown that these effects occur via SMAD-independent pathways (Murray et al., 2011, Int. J. Biochem. Cell Biol. 43:154-162). Because Sema 7a is an important mediator of TGF-β1 induced lung fibrosis that signals via noncanonical mechanisms, and because Sema 7a is also increasingly recognized as driving monocyte activation in chronic inflammatory states (Gan et al., 2011, Arthritis Rgeum. 63:2484-2494), it was explored whether Sema 7a controls M2 activation in human monocytes and in the TGF-β1 model. These data are consistent with this theory, however it was surprising to find that CD4 cells mediate these pro-fibrotic effects.

Sema 7a-expressing CD4+ cells are already known to control macrophage-mediated inflammatory effects in murine models of hypersensitivity (Suzuki et al., 2007, Nature 446:680-684). However, a role for Sema 7a expressing CD4 cells in M2 activation in lung fibrosis had been neither suggested nor explored previously. These studies revealed that Sema 7a+CD4+ cells are elevated in the blood of patients with progressive IPF and that these cells promote the induction of M2 activation and fibrogenesis in the TGF-β1 exposed murine lung. Because early studies showed that lymphocytes are not required for bleomycin-induced lung fibrosis (Helene et al., 1999, J. Leukoc. Biol. 65:187-195), current paradigms do not include a dominant role for lymphocytes in the development of fibrosis. However, more recent data indicate that over-expression of CCL18/PARC in the rodent lung induces fibrosis in a CD3-dependent manner (Luzina et al., 2009, Arthritis Rheum. 60:1530-1539) and that modulation of CD4+ lymphocyte responses attenuate collagen accumulation in animal models of renal fibrosis (Niedermeier et al., 2009, Proc. Natl. Acad. Sci. USA 106:17892-17897). In addition, CD4+ CD28+ cells have emerged as potential biomarkers of disease progression in patients with IPF (Gilani et al., 2010, PLoS One 5:e8959), though whether these cells reflect or promote disease progression remains unknown. These studies demonstrate, for the first time, that Sema 7a+CD4+ cells are elevated in the blood of IPF patients and in the TGF-β1 exposed murine lung where they participate in M2 activation and the development of fibrosis. The mechanisms through which CD4 cells could induce M2 activation and/or fibrosis are manifold, and without being bound by any particular theory, may be related to induction of pro-inflammatory cytokines via Th1 and Th17 cells, via over-exuberant Th2 responses via IL-4 and IL-13; or through abnormal dampening of immune responses that are normally induced by regulatory T cells. Although not wishing to be bound by any particular theory, it may also be that while CD4 cells are not required for fibrosis that they exert a secondary response that accentuates fibrogenesis in this model.

These data also indicate a novel association between CFL1 and M2 activation in the TGF-β1 exposed murine lung and in primary cells obtained both from IPF patients as well as normal controls. CFL1 is an actin modifying protein that allows cytoskeletal re-organization. There exist only scant literature regarding its role on macrophage activation though its increased expression by alveolar macrophages (compared to circulating monocytes), which has led to speculation that it may be involved in the monocyte to macrophage transition (Jin et al., 2004, Am. J. Respir. Cell Mol. Biol. 31:322-329). More recently CFL1 has been reported to promote macrophage activation in various disease states including fungal infection (Morato-Marques et al., 2011, J. Biol. Chem. 286:28902-28913). CFL1 activity is regulated via inhibitory phosphorylation caused by engagement of Plexin C1, the cognate receptor for Sema 7a. Although not wishing to be bound by any particular theory, the finding of increased Plexin C1 and in the circulation of IPF patients suggests a counterregulatory response that may be attempting to dampen CFL1-mediated monocyte responses. It is also noteworthy that in this study, circulating monocytes from IPF patients express at least two proteins that are traditionally associated with mature macrophages (CD206 and CFL1). Although not wishing to be bound by any particular theory, these results indicate that these monocytes may be pre-programmed to adopt their ultimate fate prior to entering the lung, either in peripheral lymph nodes or perhaps even through epigenetic alterations in the bone marrow, a finding that could have important therapeutic ramifications for the treatment of lung fibrosis. In addition, while investigation of actin-dynamics has been proposed as critically regulating the abnormalities in IPF fibroblasts, the role of actin modifying proteins in controlling the M2 activation in IPF remains largely unexplored. To the best of knowledge these are the first studies to link Sema 7a, CFL1, and alternative activation of macrophages in patients with IPF.

These findings demonstrate that Sema 7a exerts important effects on TGF-β1-induced murine lung fibrosis and M2 accumulation and these effects require the presence of CD4+ cells. Sema 7a expression is increased in the lungs and blood of IPF patients where it localizes to CD4+ lymphocytes and is most elevated in those patients with M2-skewed circulating monocytes and progressive disease. These effects are mediated at least in part via CFL1-dependent pathway. When viewed in combination, they show that a previously unrecognized relationship exists between Sema 7a, CD4 cells, M2 activation, and CFL1 and that this pathway may represent a novel therapeutic target in patients with IPF.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of inhibiting fibrosis in a mammal at risk of developing fibrosis, said method comprising administering a therapeutically effective amount of a modulator of a target to said mammal, wherein the target is selected from the group consisting of Cofilin-1 (CFL-1), Plexin C1, and any combination thereof, thereby inhibiting fibrosis in said mammal.

2. The method of claim 1, wherein said fibrosis comprises idiopathic pulmonary fibrosis.

3. The method of claim 1, wherein the modulator inhibits CFL-1.

4. The method of claim 1, wherein the modulator activates Plexin C1.

5. The method of claim 1, wherein said fibrosis comprises a pulmonary pathology selected from the list consisting of interstitial lung disease, scleroderma, radiation-induced pulmonary fibrosis, and bleomycin lung.

6. The method of claim 5, wherein said fibrosis comprises any pulmonary disorder wherein at least one of fibroproliferative matrix molecule deposition, enhanced pathological collagen accumulation, apoptosis and alveolar septal rupture with honeycombing occurs.

7. The method of claim 5, wherein said idiopathic pulmonary fibrosis is TGF-β1-induced.

8. The method of claim 1, wherein said modulator comprises an antibody, a soluble receptor of the target, an siRNA, a ribozyme, an antisense, an aptamer, a peptidomimetic, a small molecule, a soluble receptor, an agonist, or any combinations thereof.

9. The method of claim 8, wherein said antibody comprises an antibody selected from a polyclonal antibody, a monoclonal antibody, a humanized antibody, a synthetic antibody, a heavy chain antibody, a human antibody, and a biologically active fragment of an antibody.

10. The method of claim 9, wherein the biologically active fragment is a Fab fragment, and a F(ab′)₂ fragment, and combinations thereof.

11. The method of claim 9, wherein the heavy chain antibody is selected from the group consisting of a camelid antibody, a heavy chain disease antibody, and a variable heavy chain immunoglobulin.

12. The method of claim 8, wherein said antibody specifically binds to the target, a receptor of the target, a downstream effector of the target, or a functional fragment thereof.

13. The method of claim 1, wherein said mammal is a human.

14. A method of inhibiting fibrosis in a mammal diagnosed with a disease or disorder involving fibrosis, said method comprising administering a therapeutically effective amount of a modulator of a target to said mammal, wherein the target is selected from the group consisting of Cofilin-1 (CFL-1), Plexin C1, and any combination thereof, thereby inhibiting fibrosis in said mammal.

15. The method of claim 14, wherein the modulator inhibits CFL-1.

16. The method of claim 14, wherein the modulator activates Plexin C1.

17. The method of claim 14, wherein said fibrosis comprises idiopathic pulmonary fibrosis.

18. The method of claim 14, wherein said fibrosis comprises a pulmonary pathology selected from the list consisting of interstitial lung disease, scleroderma, radiation-induced pulmonary fibrosis, and bleomycin lung.

19. The method of claim 18, wherein said fibrosis comprises any pulmonary disorder wherein at least one of fibroproliferative matrix molecule deposition, enhanced pathological collagen accumulation, apoptosis and alveolar septal rupture with honeycombing occurs.

20. The method of claim 18, wherein said pulmonary fibrosis is TGF-β1-induced.

21. The method of claim 14, wherein said modulator comprises an antibody, a soluble receptor of the target, an siRNA, a ribozyme, an antisense, an aptamer, a peptidomimetic, a small molecule, a soluble receptor, an agonist or any combinations thereof.

22. The method of claim 21, wherein said antibody comprises an antibody selected from a polyclonal antibody, a monoclonal antibody, a humanized antibody, a synthetic antibody, a heavy chain antibody, a human antibody, and a biologically active fragment of an antibody, wherein the biologically active fragment is a Fab fragment, and a F(ab′)₂ fragment, and combinations thereof.

23. The method of claim 22, wherein the heavy chain antibody is selected from the group consisting of a camelid antibody, a heavy chain disease antibody, and a variable heavy chain immunoglobulin.

24. The method of claim 21, wherein said antibody specifically binds to the target, a receptor of the target, a downstream effector of the target, or functional fragments thereof.

25. The method of claim 14, wherein said mammal is a human.

26. A method of inhibiting fibrosis in a mammal at risk of developing fibrosis, said method comprising administering a therapeutically effective amount of an inhibitor for a lymphocyte expressing Sema 7a to said mammal, thereby inhibiting fibrosis in said mammal.

27. The method of claim 26, wherein the lymphocyte is a CD4 cell.
